(19) United States
(12) Patent Application Publication
(10) Pub. No.: US 2010/0004247 A1
(43) Pub. Date: Jan. 7, 2010

(54) COMBINATION COMPRISING A MEK INHIBITOR AND AN AURORA KINASE INHIBITOR 188

(75) Inventors: Kirsten Elisabeth Mundt, Rutesheim (DE); Paul David Smith, Cheshire (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/332,068

(22) Filed: Dec. 10, 2008

Related U.S. Application Data
(60) Provisional application No. 61/013,066, filed on Dec. 12, 2007.

(51) Int. Cl. A61K 31/50; A61K 31/4184; A61P 35/00

(52) U.S. Cl. 514/247; 514/394

(57) ABSTRACT

The present invention relates to a therapeutic combination comprising a MEK inhibitor and an Aurora kinase inhibitor, and to methods for the production of an anti-cancer effect in a patient, which is accordingly useful in the treatment of cancer in a patient. More specifically the present invention relates to: a therapeutic combination comprising a MEK inhibitor and an Aurora kinase inhibitor; a combination product comprising a MEK inhibitor and an Aurora kinase inhibitor, a kit of parts comprising a MEK inhibitor and an Aurora kinase inhibitor; use of a therapeutic combination, combination product or kit of parts in the treatment of cancer; a method of treating cancer comprising administering the therapeutic combination, combination product or kit of parts to a patient. The therapeutic combination and methods of the invention are also useful in the treatment of conditions in which the inhibition of MEK and/or Aurora kinase is beneficial.

This application claims the benefit under 35 U.S.C. § 119(e) of Application No. U.S. 61/031,066 filed on 12 Dec. 2007.

The present invention relates to a therapeutic combination comprising a MEK inhibitor and an Aurora kinase inhibitor, and to methods for the production of an anti-cancer effect in a patient, which is accordingly useful in the treatment of cancer in a patient. More specifically the present invention relates to: a therapeutic combination comprising a MEK inhibitor and an Aurora kinase inhibitor; a combination product comprising a MEK inhibitor and an Aurora kinase inhibitor, a kit of parts comprising a MEK inhibitor and an Aurora kinase inhibitor; use of a therapeutic combination, combination product or kit of parts in the treatment of cancer; a method of treating cancer comprising administering the therapeutic combination, combination product or kit of parts to a patient. The therapeutic combination and methods of the invention are also useful in the treatment of conditions in which the inhibition of MEK and/or Aurora kinase is beneficial.

The cell cycle describes the process of cell division, wherein a cell duplicates its DNA and separates it equally into 2 daughter cells. The final stage of chromosome segregation is called mitosis. During mitosis, the chromosomes condense and sister chromatides are aligned before they are separated, so that each nascent cell receives a full complement of the genetic material. This is accomplished by a fibrous structure, the so-called mitotic spindle, made up by microtubule polymers. Mitosis, and in particular the mitotic spindle, has been clinically validated as a drug target by compounds such as the vinca alkaloids and Taxanes. These interfere with microtubule dynamics and thus stall mitotic progression, leading to cell death. As tubulin also plays a role in many other physiological processes, the toxicity of these agents is considerable. A second generation of targeted mitotic agents is currently being developed. These agents inhibit enzymes and molecules specifically involved in mitotic regulation and may therefore be associated with less toxicity. In addition, these targets are often found to be overexpressed in cancer cells. Examples include the family of members of the Aurora kinase family, Aurora A and B, polo like kinase-1 and the kinesin like motor protein Eg5. (Jackson J R et al. “Targeted anti mitotic therapies: can we improve on tubulin agents?” Nature reviews Cancer 2007 (7) pp 107-117);

The Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization, invasion and angiogenesis (reviewed in Peyssonnaux and Eychene, Biology of the Cell, 2001, 93, 3-62). Indeed, the ras-dependent raf-MEK-MAPK cascade is one of the key signalling pathways responsible for conveying both mitogenic and invasive signals from the cell surface to the nucleus resulting in changes in gene expression and cell fate.

The Ras/Raf/MEK/ERK pathway has been reported to contribute to the tumorigenic phenotype by inducing immortalisation, growth factor-independent growth, insensitivity to growth-inhibitory signals, ability to invade and metastasise, stimulating angiogenesis and inhibition of apoptosis (reviewed in Kolch et al., Exp. Rev. Mol. Med., 25 Apr. 2002, http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway, including RAS and BRAF genes.

Accordingly, it has been recognised that an inhibitor of a protein of the MAPK kinase pathway should be of value both as an anti-proliferative, pro-apoptotic and anti-invasive agent for use in the containment and/or treatment of proliferative or invasive disease.

In addition, cellular pathways that control cell progress through the cell cycle, including mitosis, are also thought to be important in hyperproliferative diseases, which are characterised by uncontrolled cellular proliferation occurring when normal regulation of cell proliferation is lost, such as cancers.

In eukaryotes, an ordered cascade of protein phosphorylation is thought to control cell progress through the cell cycle. Several families of protein kinases that play critical roles in this cascade have been identified. The activity of many of these kinases is increased in human tumours when compared to normal tissue. This can occur by either increased levels of expression of the protein (for example as a result of gene amplification), or by changes in expression of co-activators or inhibitory proteins.

Protein kinases which play critical roles in regulating mitosis and therefore the cell cycle and which also appear to be important in oncogenesis, include the human homologues of the Drosophila aurora and S. cerevisiae Ipl1 proteins. The three human homologues of these genes aurora-A, aurora-B and aurora-C (also known as aurora2, aurora1 and aurora3 respectively) encode cell cycle regulated serine-threonine protein kinases (summarised in Adams et al., Trends in Cell Biology, 2001, 11(2): 49-54). These show a peak of expression and kinase activity through G2 and mitosis during the cell cycle. Several observations implicate the involvement of human Aurora proteins in cancer.

The aurora-A DNA is amplified and mRNA overexpressed in greater than 50% of primary human colorectal cancers, in which Aurora-A protein levels appear greatly elevated compared to adjacent normal tissue. In addition, transfection of rodent fibroblasts with human aurora-A leads to transformation, conferring the ability to grow in soft agar and form tumours in nude mice (Bischoff et al., The EMBO Journal, 1998, 17(11), 3052-3065). Other work has demonstrated that abrogation of aurora-A expression and function by antisense oligonucleotide treatment of human tumour cell lines (WO 97/22702 and WO 99/37788) leads to cell cycle arrest and exerts an antiproliferative effect in these tumour cell lines.

Aurora-B is overexpressed in cancer cells and increased levels of Aurora-B have been shown to correlate with advanced stages of colorectal cancer (Katayama et al., J. Natl Cancer Inst., 1999, 91, 1160). Furthermore, it has been reported that overexpression of aurora-B induces aneuploidy and that cells overexpressing aurora-B form more aggressive tumours that develop metastases (Ota T. et al., Cancer Res., 2002, 62, 5168-5177).

With regard to aurora-C, its expression is thought to be restricted to the testis although it has been found to be overexpressed in various cancer lines (Katayama H et al., Cancer and Metastasis Reviews, 2003, 22: 451-464).

Additionally, small molecule inhibitors of Aurora-A and Aurora-B have been demonstrated to have an antiproliferative effect in human tumour cells (Keen et al. 2001, Poster #2455, American Association of Cancer Research annual meeting), as has selective abrogation of aurora-B expression alone by siRNA treatment (Ditchfield et al., J. Cell Biology, 2003, 161(2), 267-280). This indicates that inhibition of the function of aurora-A and/or aurora-B will have an antiproliferative effect that may be useful in the treatment of human tumours and other hyperproliferative disease.

Mitosis and the cell cycle play an intrinsic role in the proliferation of all tumour cells. Accordingly it has been recognised that inhibition of mitosis and therefore the cell cycle, as a therapeutic approach to hyperproliferative diseases such as cancer, would be active in proliferating tumour cells. Approaches directed at specific signalling molecules, such as MEK, would be expected to be effective in the subset of tumour cells which express, and are at least partly dependent on, the Raf-MEK-MAPK cascade.

Inhibition of MEK1/2 in cultured tumour cells frequently causes an arrest in the G1 phase of the cell cycle and over time this results in a reduction in the percentage of cells in other phases of the cell cycle relative to untreated cells. These other phases of the cell cycle are S-phase (period in which DNA synthesis occurs) and mitosis (period in which cell division occurs). When the MEK inhibitor is removed from the culture medium, cells arrested in the G1 phase of the cell cycle begin to move into S-phase and then mitosis in a synchronous fashion such that at certain periods following release from MEK inhibition there is an increase in the percentage of cells in S-phase and mitosis of the cell cycle relative to untreated cells. Therefore MEK inhibition followed by release from MEK inhibition has the potential to enrich the cell population for those in mitosis and therefore those sensitive to mitosis inhibitors, such as an Aurora kinase inhibitor.

The present invention relates to a therapeutic combination comprising a MEK inhibitor, or a pharmaceutically acceptable salt thereof, and an Aurora kinase inhibitor, or a pharmaceutically acceptable salt thereof. The therapeutic combination is useful in a method for the production of an anti-cancer effect in a patient, which is accordingly useful in the treatment of cancer in a patient.

The therapeutic combination may be in the form of a combination product comprising a MEK inhibitor, or a pharmaceutically acceptable salt thereof, and an Aurora kinase inhibitor, or a pharmaceutically acceptable salt thereof. The therapeutic combination may comprise a kit of parts comprising separate formulations of a MEK inhibitor, or a pharmaceutically acceptable salt thereof, and an Aurora kinase inhibitor, or a pharmaceutically acceptable salt thereof. The separate formulations of a MEK inhibitor, or a pharmaceutically acceptable salt thereof, and an Aurora kinase inhibitor, or a pharmaceutically acceptable salt thereof, may be administered sequentially, separately and/or simultaneously. It will be apparent to the skilled person that the separate formulations of a MEK inhibitor, or a pharmaceutically acceptable salt thereof, and an Aurora kinase inhibitor, or a pharmaceutically acceptable salt thereof, can be administered simultaneously (optionally repeatedly). It will also be apparent to the skilled person that the separate formulations of a MEK inhibitor, or a pharmaceutically acceptable salt thereof, and an Aurora kinase inhibitor, or a pharmaceutically acceptable salt thereof, can be administered sequentially (optionally repeatedly). It will also be apparent to the skilled person that the separate formulations of a MEK inhibitor, or a pharmaceutically acceptable salt thereof, and an Aurora kinase inhibitor, or a pharmaceutically acceptable salt thereof, of the therapeutic combination can be administered separately (optionally repeatedly). The skilled person will also understand that where the separate formulations of a MEK inhibitor, or a pharmaceutically acceptable salt thereof, and an Aurora kinase inhibitor, or a pharmaceutically acceptable salt thereof, are administered sequentially or serially that this could be by administration of a MEK inhibitor, or a pharmaceutically acceptable salt thereof, followed by an Aurora kinase inhibitor, or a pharmaceutically acceptable salt thereof, or administration of an Aurora kinase inhibitor, or a pharmaceutically acceptable salt thereof, followed by a MEK inhibitor, or a pharmaceutically acceptable salt thereof. Furthermore, the separate formulations of a MEK inhibitor, or a pharmaceutically acceptable salt thereof, and an Aurora kinase inhibitor, or a pharmaceutically acceptable salt thereof, may be administered in alternative dosing patterns. Where the separate formulations of a MEK inhibitor, or a pharmaceutically acceptable salt thereof, and an Aurora kinase inhibitor, or a pharmaceutically acceptable salt thereof, are administered sequentially or separately, the delay in administering the second formulation should not be such as to lose the beneficial effect of the therapeutic combination. Thus, a therapeutic combination comprising a MEK inhibitor, or a pharmaceutically-acceptable salt thereof, and an Aurora kinase inhibitor, or a pharmaceutically-acceptable salt thereof, could be used sequentially, separately and/or simultaneously in the treatment of cancer.

In one aspect of the present invention there is provided a therapeutic combination comprising

a MEK inhibitor, or a pharmaceutically acceptable salt thereof, and

an Aurora kinase inhibitor, or a pharmaceutically acceptable salt thereof.

In one embodiment of the present invention there is provided a therapeutic combination comprising

a MEK inhibitor, or a pharmaceutically acceptable salt thereof, and

an Aurora kinase inhibitor, or a pharmaceutically acceptable salt thereof, in association with a pharmaceutically acceptable adjuvant, diluent or carrier.

In one embodiment of the present invention there is provided a therapeutic combination comprising

a MEK inhibitor, or a pharmaceutically acceptable salt thereof, in association with a pharmaceutically acceptable adjuvant, diluent or carrier and

an Aurora kinase inhibitor, or a pharmaceutically acceptable salt thereof, in association with a pharmaceutically acceptable adjuvant, diluent or carrier

Surprisingly, we have found that sequential inhibition of Aurora kinase followed by inhibition of MEK, is particularly beneficial and yields synergistic inhibition of tumour cell growth in vivo, in comparison with inhibition of Aurora kinase or inhibition of MEK alone. It is proposed that sequential inhibition of Aurora kinase followed by inhibition of MEK will also lead to greater inhibition of tumour cell growth than that which would be achieved by the inhibition of either Aurora kinase or MEK alone.

In a particular aspect of the present invention there is provided a therapeutic combination comprising

a MEK inhibitor, or a pharmaceutically acceptable salt thereof, and

an Aurora kinase inhibitor, or a pharmaceutically acceptable salt thereof,

wherein one or more doses of the Aurora kinase inhibitor, or a pharmaceutically acceptable salt thereof, are administered prior to, or simultaneously with, the administration of one or more doses of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein multiple doses of the Aurora kinase inhibitor, or a pharmaceutically acceptable salt thereof, are administered prior to, or simultaneously with, the administration of multiple doses of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein multiple doses of the Aurora kinase inhibitor, or a pharmaceutically acceptable salt thereof, are administered prior to, or simultaneously with, the administration of a dose of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein a dose of the Aurora kinase inhibitor, or a pharmaceutically acceptable salt thereof, is administered prior to, or simultaneously with, the administration of multiple doses of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein a dose of the Aurora kinase inhibitor, or a pharmaceutically acceptable salt thereof, is administered prior to, or simultaneously with, the administration of a dose of the MEK inhibitor, or a pharmaceutically acceptable salt thereof.

In a particular embodiment of the present invention there is provided a therapeutic combination comprising

a MEK inhibitor, or a pharmaceutically acceptable salt thereof, and

an Aurora kinase inhibitor, or a pharmaceutically acceptable salt thereof,

wherein one or more doses of the Aurora kinase inhibitor, or a pharmaceutically acceptable salt thereof, are administered prior to the administration of one or more doses of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein multiple doses of the Aurora kinase inhibitor, or a pharmaceutically acceptable salt thereof, are administered prior to the administration of multiple doses of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein multiple doses of the Aurora kinase inhibitor, or a pharmaceutically acceptable salt thereof, are administered prior to the administration of a dose of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein a dose of the Aurora kinase inhibitor, or a pharmaceutically acceptable salt thereof, is administered prior to the administration of multiple doses of the MEK inhibitor, or a pharmaceutically acceptable salt thereof.

In another embodiment of the present invention there is provided a therapeutic combination comprising:

a MEK inhibitor, or a pharmaceutically acceptable salt thereof, and

an Aurora kinase inhibitor, or a pharmaceutically acceptable salt thereof,

wherein a dose of the Aurora kinase inhibitor, or a pharmaceutically acceptable salt thereof, is administered prior to the administration of a dose of the MEK inhibitor, or a pharmaceutically acceptable salt thereof.

In another aspect of the present invention there is provided a combination product comprising

a MEK inhibitor, or a pharmaceutically acceptable salt thereof, and

an Aurora kinase inhibitor, or a pharmaceutically acceptable salt thereof, in association with a pharmaceutically acceptable adjuvant, diluent or carrier.

The combination product may comprise separate formulations of

a MEK inhibitor, or a pharmaceutically acceptable salt thereof, in association with a pharmaceutically acceptable adjuvant, diluent or carrier and

an Aurora kinase inhibitor, or a pharmaceutically acceptable salt thereof, in association with a pharmaceutically acceptable adjuvant, diluent or carrier. Alternatively, the combination product may comprise a combined formulation of

a MEK inhibitor, or a pharmaceutically acceptable salt thereof, and

an Aurora kinase inhibitor, or a pharmaceutically acceptable salt thereof, in association with a pharmaceutically acceptable adjuvant, diluent or carrier.

In a particular embodiment of the invention, one or more doses of the Aurora kinase inhibitor, or a pharmaceutically acceptable salt thereof, are administered prior to, or simultaneously with, the administration of one or more doses of the MEK inhibitor, or a pharmaceutically acceptable salt thereof.

In another embodiment of the invention, multiple doses of the Aurora kinase inhibitor, or a pharmaceutically acceptable salt thereof, are administered prior to, or simultaneously with, the administration of multiple doses of the MEK inhibitor, or a pharmaceutically acceptable salt thereof.

In another embodiment of the invention, multiple doses of the Aurora kinase inhibitor, or a pharmaceutically acceptable salt thereof, are administered prior to, or simultaneously with, the administration of a dose of the MEK inhibitor, or a pharmaceutically acceptable salt thereof.

In another embodiment of the invention, a dose of the Aurora kinase inhibitor, or a pharmaceutically acceptable salt thereof, is administered prior to, or simultaneously with, the administration of multiple doses of the MEK inhibitor, or a pharmaceutically acceptable salt thereof.

In another embodiment of the invention, a dose of the Aurora kinase inhibitor, or a pharmaceutically acceptable salt thereof, is administered prior to, or simultaneously with, the administration of a dose of the MEK inhibitor, or a pharmaceutically acceptable salt thereof.

In a particular embodiment of the invention, one or more doses of the Aurora kinase inhibitor, or a pharmaceutically acceptable salt thereof, are administered prior to the administration of one or more doses of the MEK inhibitor, or a pharmaceutically acceptable salt thereof.

In another embodiment of the invention, multiple doses of the Aurora kinase inhibitor, or a pharmaceutically acceptable salt thereof, are administered prior to the administration of multiple doses of the MEK inhibitor, or a pharmaceutically acceptable salt thereof.

In another embodiment of the invention, multiple doses of the Aurora kinase inhibitor, or a pharmaceutically acceptable salt thereof, are administered prior to the administration of a dose of the MEK inhibitor, or a pharmaceutically acceptable salt thereof.

In another embodiment of the invention, a dose of the Aurora kinase inhibitor, or a pharmaceutically acceptable salt thereof, is administered prior to the administration of multiple doses of the MEK inhibitor, or a pharmaceutically acceptable salt thereof.

In another embodiment of the invention, a dose of the Aurora kinase inhibitor, or a pharmaceutically acceptable salt thereof, is administered prior to the administration of a dose of the MEK inhibitor, or a pharmaceutically acceptable salt thereof.

In another aspect there is provided a kit of parts comprising the following components
a MEK inhibitor, or a pharmaceutically acceptable salt thereof, in association with a pharmaceutically acceptable adjuvant, diluent or carrier; and
an Aurora kinase inhibitor, or a pharmaceutically acceptable salt thereof, in association with a pharmaceutically acceptable adjuvant, diluent or carrier.

In one embodiment of the invention the kit of parts comprising the following components
a MEK inhibitor, or a pharmaceutically acceptable salt thereof, in association with a pharmaceutically acceptable adjuvant, diluent or carrier; and
an Aurora kinase inhibitor, or a pharmaceutically acceptable salt thereof, in association with a pharmaceutically acceptable adjuvant, diluent or carrier,

wherein the components are provided in a form which is suitable for sequential, separate and/or simultaneous administration.

In one embodiment the kit of parts comprises
a first container comprising a MEK inhibitor, or a pharmaceutically acceptable salt thereof, in association with a pharmaceutically acceptable adjuvant, diluent or carrier; and
a second container comprising an Aurora kinase inhibitor, or a pharmaceutically acceptable salt thereof, in association with a pharmaceutically acceptable adjuvant, diluent or carrier, and
   a container means for containing said first and second containers.

In one embodiment the kit of parts further comprises instructions to administer the components sequentially, separately and/or simultaneously. Suitably, the instructions describe the administration sequence as described herein. In a particular embodiment the instructions indicate that the therapeutic combination can be used in the treatment of cancer. In one embodiment the instructions indicate that the therapeutic combination can be used in the treatment of conditions in which the inhibition of MEK and/or Aurora kinase is beneficial.

In another aspect of the present invention there is provided a method of treating
cancer, or
conditions in which the inhibition of MEK and/or Aurora kinase is beneficial,

comprising administration of a therapeutically effective amount of a MEK inhibitor, or a pharmaceutically acceptable salt thereof, in combination with an Aurora kinase inhibitor, or a pharmaceutically acceptable salt thereof, to a patient having, or suspected of having, cancer.

In another aspect of the present invention there is provided a method of treating cancer, which comprises administration of a therapeutically effective amount of a therapeutic combination, combination product or kit of parts as hereinbefore defined, to a patient having, or suspected of having, cancer.

In another aspect of the present invention there is provided a method of treating conditions in which the inhibition of MEK and/or Aurora kinase is beneficial, which comprises administration of a therapeutically effective amount of a therapeutic combination, combination product or kit of parts as hereinbefore defined, to a patient.

In one embodiment there is provided a method of treating
cancer, or
conditions in which the inhibition of MEK and/or Aurora kinase is beneficial,

wherein one or more doses of the Aurora kinase inhibitor, or a pharmaceutically acceptable salt thereof, are administered prior to, or simultaneously with, the administration of one or more doses of the MEK inhibitor, or a pharmaceutically acceptable salt thereof.

In one embodiment there is provided a method of treating
cancer, or
conditions in which the inhibition of MEK and/or Aurora kinase is beneficial,

wherein multiple doses of the Aurora kinase inhibitor, or a pharmaceutically acceptable salt thereof, are administered prior to, or simultaneously with, the administration of multiple doses of the MEK inhibitor, or a pharmaceutically acceptable salt thereof.

In one embodiment there is provided a method of treating
cancer, or
conditions in which the inhibition of MEK and/or Aurora kinase is beneficial,

wherein multiple doses of the Aurora kinase inhibitor, or a pharmaceutically acceptable salt thereof, are administered prior to, or simultaneously with, the administration of a dose of the MEK inhibitor, or a pharmaceutically acceptable salt thereof.

In one embodiment there is provided a method of treating
cancer, or
conditions in which the inhibition of MEK and/or Aurora kinase is beneficial,

wherein a dose of the Aurora kinase inhibitor, or a pharmaceutically acceptable salt thereof, is administered prior to, or simultaneously with, the administration of multiple doses of the MEK inhibitor, or a pharmaceutically acceptable salt thereof.

In one embodiment there is provided a method of treating
cancer, or
conditions in which the inhibition of MEK and/or Aurora kinase is beneficial,

wherein a dose of the Aurora kinase inhibitor, or a pharmaceutically acceptable salt thereof, is administered prior to, or simultaneously with, the administration of a dose of the MEK inhibitor, or a pharmaceutically acceptable salt thereof.

In one embodiment the method of treatment additionally comprises selecting a patient in need of MEK and/or Aurora kinase inhibition.

In one embodiment the method of treatment additionally comprises selecting a patient in need of treatment for cancer.

As previously described above, it is beneficial if the method of treatment of cancer, or conditions in which the inhibition of MEK and/or Aurora kinase is beneficial, comprises the steps of administering a dose of the Aurora kinase inhibitor, or a pharmaceutically acceptable salt thereof, prior to, or simultaneously with, the administration of a dose of the MEK inhibitor, or a pharmaceutically acceptable salt thereof. It is particularly beneficial if the method of treatment of cancer or conditions in which the inhibition of MEK and/or Aurora kinase is beneficial comprises the steps of administering one or more doses of the Aurora kinase inhibitor, or a pharmaceutically acceptable salt thereof, prior to the administration of one or more doses of the MEK inhibitor, or a pharmaceutically acceptable salt thereof. In one embodiment a dose of the MEK inhibitor, or a pharmaceutically acceptable salt thereof, may be administered during (but after commencement of) the administration of a dose of the Aurora kinase inhibitor, or a pharmaceutically acceptable salt thereof. In another embodiment a dose of the MEK inhibitor, or a pharmaceutically acceptable salt thereof, may be administered immediately following the completion of the administration of one or more doses of the Aurora kinase inhibitor, or a pharmaceutically acceptable salt thereof. In another embodiment there may be an interval between the administration of one or more doses of the Aurora kinase inhibitor, or a pharmaceutically acceptable salt thereof, and the administration of a dose of the MEK inhibitor, or a pharmaceutically acceptable salt thereof.

In another aspect of the present invention there is provided use of a therapeutic combination, combination product or kit of parts as hereinbefore defined in the manufacture of a medicament.

In another aspect of the present invention there is provided use of a MEK inhibitor, or a pharmaceutically acceptable salt thereof, in combination with an Aurora kinase inhibitor, or a pharmaceutically acceptable salt thereof, in the manufacture of a medicament.

In one embodiment there is provided use of a therapeutic combination, combination product or kit of parts as hereinbefore defined in the manufacture of a medicament for the treatment of cancer.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament for the treatment of cancer.

In another aspect of the present invention there is provided use of a therapeutic combination, combination product or kit of parts as hereinbefore defined in the manufacture of a medicament for the treatment of conditions in which the inhibition of MEK and/or Aurora kinase is beneficial.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament for the treatment of conditions in which the inhibition of MEK and/or Aurora kinase is beneficial.

In one embodiment there is provided use of a therapeutic combination, combination product or kit of parts as hereinbefore defined in the manufacture of a medicament for the treatment of
cancer, or
conditions in which the inhibition of MEK and/or Aurora kinase is beneficial,

wherein one or more doses of the Aurora kinase inhibitor, or a pharmaceutically acceptable salt thereof, are administered prior to, or simultaneously with, the administration of one or more doses of the MEK inhibitor, or a pharmaceutically acceptable salt thereof.

In one embodiment there is provided use of a therapeutic combination, combination product or kit of parts as hereinbefore defined in the manufacture of a medicament for the treatment of
cancer, or
conditions in which the inhibition of MEK and/or Aurora kinase is beneficial,

wherein multiple doses of the Aurora kinase inhibitor, or a pharmaceutically acceptable salt thereof, are administered prior to, or simultaneously with, the administration of multiple doses of the MEK inhibitor, or a pharmaceutically acceptable salt thereof.

In one embodiment there is provided use of a therapeutic combination, combination product or kit of parts as hereinbefore defined in the manufacture of a medicament for the treatment of
cancer, or
conditions in which the inhibition of MEK and/or Aurora kinase is beneficial,

wherein multiple doses of the Aurora kinase inhibitor, or a pharmaceutically acceptable salt thereof, are administered prior to, or simultaneously with, the administration of a dose of the MEK inhibitor, or a pharmaceutically acceptable salt thereof.

In one embodiment there is provided use of a therapeutic combination, combination product or kit of parts as hereinbefore defined in the manufacture of a medicament the treatment of
cancer, or
conditions in which the inhibition of MEK and/or Aurora kinase is beneficial,

wherein a dose of the Aurora kinase inhibitor, or a pharmaceutically acceptable salt thereof, is administered prior to, or simultaneously with, the administration of multiple doses of the MEK inhibitor, or a pharmaceutically acceptable salt thereof.

In one embodiment there is provided use of a therapeutic combination, combination product or kit of parts as hereinbefore defined in the manufacture of a medicament for the treatment of
cancer, or
conditions in which the inhibition of MEK and/or Aurora kinase is beneficial,

wherein a dose of the Aurora kinase inhibitor, or a pharmaceutically acceptable salt thereof, is administered prior to, or simultaneously with, the administration of a dose of the MEK inhibitor, or a pharmaceutically acceptable salt thereof.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament for the treatment of
cancer, or
conditions in which the inhibition of MEK and/or Aurora kinase is beneficial,

wherein one or more doses of the Aurora kinase inhibitor, or a pharmaceutically acceptable salt thereof, are administered prior to, or simultaneously with, the administration of one or more doses of the MEK inhibitor, or a pharmaceutically acceptable salt thereof.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament for the treatment of
cancer, or
conditions in which the inhibition of MEK and/or Aurora kinase is beneficial,

wherein multiple doses of the Aurora kinase inhibitor, or a pharmaceutically acceptable salt thereof, are administered prior to, or simultaneously with, the administration of multiple doses of the MEK inhibitor, or a pharmaceutically acceptable salt thereof.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament for the treatment of
cancer, or
conditions in which the inhibition of MEK and/or Aurora kinase is beneficial,

wherein multiple doses of the Aurora kinase inhibitor, or a pharmaceutically acceptable salt thereof, are administered prior to, or simultaneously with, the administration of a dose of the MEK inhibitor, or a pharmaceutically acceptable salt thereof.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament the treatment of
cancer, or
conditions in which the inhibition of MEK and/or Aurora kinase is beneficial,

wherein a dose of the Aurora kinase inhibitor, or a pharmaceutically acceptable salt thereof, is administered prior to, or simultaneously with, the administration of multiple doses of the MEK inhibitor, or a pharmaceutically acceptable salt thereof.

In one embodiment there is provided use of a MEK inhibitor, or a pharmaceutically acceptable salt thereof, in combination with an Aurora kinase inhibitor, or a pharmaceutically acceptable salt thereof, in the manufacture of a medicament for the treatment of
cancer, or
conditions in which the inhibition of MEK and/or Aurora kinase is beneficial,

wherein a dose of the Aurora kinase inhibitor, or a pharmaceutically acceptable salt thereof, is administered prior to, or simultaneously with, the administration of a dose of the MEK inhibitor, or a pharmaceutically acceptable salt thereof.

In one embodiment the use additionally comprises selecting a patient in need of MEK and/or Aurora kinase inhibition.

In one embodiment the use additionally comprises selecting a patient in need of treatment for cancer.

In another aspect of the present invention there is provided a therapeutic combination, combination product or kit of parts as hereinbefore described for use as a medicament.

In another aspect of the present invention there is provided a therapeutic combination comprising a MEK inhibitor, or a pharmaceutically acceptable salt thereof, and an Aurora kinase inhibitor, or a pharmaceutically acceptable salt thereof, for use as a medicament.

In another aspect of the present invention there is provided a therapeutic combination, combination product or kit of parts as hereinbefore described for use in the treatment of cancer.

In another aspect of the present invention there is provided a therapeutic combination comprising a MEK inhibitor, or a pharmaceutically acceptable salt thereof, and an Aurora kinase inhibitor, or a pharmaceutically acceptable salt thereof, for use in the treatment of cancer.

In another aspect of the present invention there is provided a therapeutic combination, combination product or kit of parts as hereinbefore described for use in the treatment of conditions in which the inhibition of MEK and/or Aurora kinase is beneficial.

In one embodiment there is provided a therapeutic combination, combination product or kit of parts as hereinbefore defined for use in the treatment of
cancer, or
conditions in which the inhibition of MEK and/or Aurora kinase is beneficial,

wherein one or more doses of the Aurora kinase inhibitor, or a pharmaceutically acceptable salt thereof, are administered prior to the administration of one or more doses of the MEK inhibitor, or a pharmaceutically acceptable salt thereof.

In one embodiment there is provided a therapeutic combination, combination product or kit of parts as hereinbefore defined for use in the treatment of
cancer, or
conditions in which the inhibition of MEK and/or Aurora kinase is beneficial,

wherein multiple doses of the Aurora kinase inhibitor, or a pharmaceutically acceptable salt thereof, are administered prior to the administration of multiple doses of the MEK inhibitor, or a pharmaceutically acceptable salt thereof.

In one embodiment there is provided a therapeutic combination, combination product or kit of parts as hereinbefore defined for use in the treatment of
cancer, or
conditions in which the inhibition of MEK and/or Aurora kinase is beneficial,

wherein multiple doses of the Aurora kinase inhibitor, or a pharmaceutically acceptable salt thereof, are administered prior to the administration of a dose of the MEK inhibitor, or a pharmaceutically acceptable salt thereof.

In one embodiment there is provided a therapeutic combination, combination product or kit of parts as hereinbefore defined for use in the treatment of
cancer, or
conditions in which the inhibition of MEK and/or Aurora kinase is beneficial,

wherein a dose of the Aurora kinase inhibitor, or a pharmaceutically acceptable salt thereof, is administered prior to the administration of multiple doses of the MEK inhibitor, or a pharmaceutically acceptable salt thereof.

In one embodiment there is provided a therapeutic combination, combination product or kit of parts as hereinbefore defined for use in the treatment of
cancer, or
conditions in which the inhibition of MEK and/or Aurora kinase is beneficial,

wherein a dose of the Aurora kinase inhibitor, or a pharmaceutically acceptable salt thereof, is administered prior to the administration of a dose of the MEK inhibitor, or a pharmaceutically acceptable salt thereof.

In one embodiment the use additionally comprises selecting a patient in need of MEK and/or Aurora kinase inhibition.

In one embodiment the use additionally comprises selecting a patient in need of treatment for cancer.

In one embodiment there is provided a method or use as described hereinabove wherein the patient is not resistant to MEK inhibition.

In one embodiment there is provided a method or use as described hereinabove wherein the patient is not resistant to Aurora kinase inhibition.

In one embodiment there is provided a method or use as described hereinabove wherein the patient's tumour carries any one or more of, a BRaf mutation or mutations, a KRas mutation or mutations, a NRas mutation or mutations, and/or a HRaf mutation or mutations.

For the avoidance of doubt, by “administered prior to” we mean that administration of one or more doses of the Aurora kinase inhibitor, or a pharmaceutically acceptable salt thereof, are commenced before administration of one or more doses of the MEK inhibitor, or a pharmaceutically acceptable salt thereof. Therefore, the expression “administered prior to” encompasses a situation where one or more doses of the Aurora kinase inhibitor, or a pharmaceutically acceptable salt thereof, are administered first and this is followed by the administration of one or more doses of the MEK inhibitor, or a pharmaceutically acceptable salt thereof. A dose of the MEK inhibitor, or a pharmaceutically acceptable salt thereof, may be administered during (but after commencement of) the administration of a dose of the Aurora kinase inhibitor, or a pharmaceutically acceptable salt thereof. Alternatively, a dose of the MEK inhibitor, or a pharmaceutically acceptable salt thereof, may be administered immediately following the completion of the administration of one or more doses of the Aurora kinase inhibitor, or a pharmaceutically acceptable salt thereof. Furthermore, there may be an interval between the administration of one or more doses of the Aurora kinase inhibitor, or a pharmaceutically acceptable salt thereof, and the administration of a dose of the MEK inhibitor, or a pharmaceutically acceptable salt thereof. The interval can be calculated such as to optimise anti-cancer effects of the inhibitors and to minimise undesirable interaction between the inhibitors. Undesirable interaction between the inhibitors may be a reduced efficacy in comparison with either inhibitor used alone, or an increased toxicity in comparison with either inhibitor used alone.

By “simultaneously” we mean that a dose of the Aurora kinase inhibitor, or a pharmaceutically acceptable salt thereof, is administered at the same time as the administration of a dose of the MEK inhibitor, or a pharmaceutically acceptable salt thereof.

In a particular embodiment of the invention, the interval between administration of one or more doses of the Aurora kinase inhibitor, or a pharmaceutically acceptable salt thereof, and administration of a dose of the MEK inhibitor, or a pharmaceutically acceptable salt thereof, is 0 hours to 2 weeks, for example 12 hours, 24 hours, 1 day, 2, 3, 4, 5, 6, 7, 8, 9, 10, 11, 12, 13 or 14 days.

In a further embodiment the interval between administration of one or more doses of the Aurora kinase inhibitor, or a pharmaceutically acceptable salt thereof, and administration of a dose of the MEK inhibitor, or a pharmaceutically acceptable salt thereof, is 0.5 to 5 days.

In a further embodiment the interval between administration of one or more doses of the Aurora kinase inhibitor, or a pharmaceutically acceptable salt thereof, and administration of a dose of the MEK inhibitor, or a pharmaceutically acceptable salt thereof, is 1 day.

In a further embodiment the interval between administration of one or more doses of the Aurora kinase inhibitor, or a pharmaceutically acceptable salt thereof, and administration of a dose of the MEK inhibitor, or a pharmaceutically acceptable salt thereof, is 24 hours.

It will be appreciated that the administration of one or more doses of an Aurora kinase inhibitor, or a pharmaceutically acceptable salt thereof, and the administration of one or more doses of a MEK inhibitor, or a pharmaceutically acceptable salt thereof, may be repeated numerous times during a treatment regime.

Therefore, following administration of a dose or multiple doses of the MEK inhibitor, or a pharmaceutically acceptable salt thereof, the Aurora kinase inhibitor, or a pharmaceutically acceptable salt thereof, can be administered again prior to, or simultanesously with, a dose of the MEK inhibitor, or a pharmaceutically acceptable salt thereof. In one embodiment this subsequent administration of the Aurora kinase inhibitor, or a pharmaceutically acceptable salt thereof, immediately follows administration of a dose of the MEK inhibitor, or a pharmaceutically acceptable salt thereof. In an alternative embodiment there is an interval following administration of a dose of the MEK inhibitor, or a pharmaceutically acceptable salt thereof, prior to the subsequent administration of the Aurora kinase inhibitor, or a pharmaceutically acceptable salt thereof. In one embodiment the interval between administration of a dose of the MEK inhibitor, or a pharmaceutically acceptable salt thereof, and subsequent administration of a dose of the Aurora kinase inhibitor, or a pharmaceutically acceptable salt thereof, is greater than 1 day.

In one embodiment the MEK inhibitor is a small molecular weight compound. In one embodiment the MEK inhibitor is selected from any one of an ATP-competitive MEK inhibitor, a non-ATP competitive MEK inhibitor, or an ATP-uncompetitive MEK inhibitor. International Patent Publication Number WO2003/077914 discloses the compound 6-(4-Bromo-2-chloro-phenylamino)-7-fluoro-3-methyl-3H-benzoimidazole-5-carboxylic acid (2-hydroxy-ethoxy)-amide which is referred to herein as AZD6244. In one embodiment the MEK inhibitor is selected from any one of AZD6244, 2-(2-fluoro-4-iodophenylamino)-N-(2-hydroxyethoxy)-1,5-dimethyl-6-oxo-1,6-dihydropyridine-3-carboxamide, 4-(4-Bromo-2-fluorophenylamino)-N-(2-hydroxyethoxy)-1,5-dimethyl-6-oxo-1,6-dihydropyridazine-3-carboxamide, PD-0325901 (Pfizer), PD-184352 (Pfizer), XL-518 (Exelixis), AR-119 (Ardea Biosciences, Valeant Pharmaceuticals), AS-701173 (Merck Serono), AS-701255 (Merck Serono), 360770-54-3 (Wyeth). In one embodiment the MEK inhibitor is selected from AZD6244, 2-(2-fluoro-4-iodophenylamino)-N-(2-hydroxyethoxy)-1,5-dimethyl-6-oxo-1,6-dihydropyridine-3-carboxamide or 4-(4-Bromo-2-fluorophenylamino)-N-(2-hydroxyethoxy)-1,5-dimethyl-6-oxo-1,6-dihydropyridazine-3-carboxamide as described below.

In one embodiment the MEK inhibitor is selected from AZD6244, 4-(4-Bromo-2-fluorophenylamino)-N-(2-hydroxyethoxy)-1,5-dimethyl-6-oxo-1,6-dihydropyridazine-3-carboxamide or 2-(2-fluoro-4-iodophenylamino)-N-(2-hydroxyethoxy)-1,5-dimethyl-6-oxo-1,6-dihydropyridine-3-carboxamide, as described below.

In one embodiment the MEK inhibitor is selected from AZD6244 or a pharmaceutically acceptable salt thereof. In one embodiment the MEK inhibitor is AZD6244 hydrogen sulphate salt. AZD6244 hydrogen sulphate salt may be synthesised according to the process described in International Patent Publication Number WO2007/076245.

In one embodiment the MEK inhibitor is selected from 6-(4-Bromo-2-chloro-phenylamino)-7-fluoro-3-methyl-3H-benzoimidazole-5-carboxylic acid (2-hydroxy-ethoxy)-amide or a pharmaceutically acceptable salt thereof. In one embodiment the MEK inhibitor is 6-(4-Bromo-2-chloro-phenylamino)-7-fluoro-3-methyl-3H-benzoimidazole-5-carboxylic acid (2-hydroxy-ethoxy)-amide hydrogen sulphate salt. 6-(4-Bromo-2-chloro-phenylamino)-7-fluoro-3-methyl-3H-benzoimidazole-5-carboxylic acid (2-hydroxy-ethoxy)-amide hydrogen sulphate salt may be synthesised according to the process described in International Patent Publication Number WO2007/076245.

In another embodiment the MEK inhibitor may inhibit gene expression, for example by interfering with mRNA stability or translation. In one embodiment the MEK inhibitor is selected from small interfering RNA (siRNA), which is sometimes known as short interfering RNA or silencing RNA, or short hairpin RNA (shRNA), which is sometimes known as small hairpin RNA.

In one embodiment the Aurora kinase inhibitor is selected from any one of MLN8054 (Millennium), MLN-8237 (Millennium), ENMD-981693 (EntreMed), MP235+(Montigen), MP529 (Montigen), AX39459 (GPC/AXXIMA), Aur A (University Arizona), Aur A (Aventis), Aur B (Inploid/NCE/Wolfson), Aur B (GSK), Aur B (Boehringer Ingelheim), PH739358 (Nerviano), MK0457/VX-680 (Merck % Vertex), AT9283 (Astex), R763 (Rigel/Serono, MK6592/VX-667 (Merck/Vertex), CYC116 (Cyclacel), SNS-314 (Sunesis), AKI-001 (Roche, Genentech), AKI (Elara Pharmaceuticals), AKI (Avalon Pharmaceuticals), A-60/EA-19/E2B8 (Telik Inc.), BMS-739562 (Bristol-Myers Squibb), AKI (Sarem Holdings), GSK-1070916A (GlaxoSmithKline), S-081/S-091 (Sentinel Oncology), MK-615 (Japon Apricot), PF-3814735 (Pfizer), Danusertib (Pfizer), EN-640402 (Amgen), TTP-607 (TransTech Pharma), VX-689 (Vertex), VE-465 (Vertex), SU-6668, SGI-529 (University of Arizona), KW-2449 (Kyowa-Hakko), XL 228 (Exelixis). AB-038 (Ambit), BI 811283 (Boehringer Ingelheim), CHR3520 (Chroma) and RO4612910 (Roche).

In one embodiment the Aurora kinase inhibitor is selected from any one of the inhibitors of Aurora kinase described in International Patent Publication Numbers WO2003/055491, WO2004/058781, WO2004/058781 and WO2006/129064. WO2004/058781 in particular discloses the compound 2-{[3-({4-[(5-{2-[(3-fluorophenyl)amino]-2-oxoethyl}-1H-pyrazol-3-yl)amino]quinazolin-7-yl}oxy)propyl](ethyl)amino}ethyl dihydrogen phosphate, which is referred to herein as AZD1152. AZD1152 is a pro-drug that is rapidly and completely converted (in human plasma) to the active moiety which possesses the following formula 2-{3-[(7-{3-[ethyl(2-hydroxyethyl)amino]propoxy}quinazolin-4-yl)amino]-1H-pyrazol-5-yl}-N-(3-fluorophenyl)acetamide, referred to herein as AZD1152 HQPA. AZD1152 HQPA is an ATP-competitive and reversible inhibitor of the aurora kinases with potent activity against aurora A, B-INCENP and C-INCENP (Ki's 1369±419.2 nM, 0.359±0.386 nM and 17.03±12.2 nM respectively). AZD1152 has been found to inhibit tumour growth in a panel of human colorectal (SW620, HCT116, Colo205) and lung (A549, Calu-6) tumour xenografts with statistical significance. A maleate co-crystal of AZD1152 is described in International Patent Publication Number WO2007/132227.

In one embodiment the Aurora kinase inhibitor is AZD1152, or a pharmaceutically acceptable salt thereof.

In one embodiment the Aurora kinase inhibitor is 2-{[3-({4-[(5-{2-[(3-fluorophenyl)amino]-2-oxoethyl}-1H-pyrazol-3-yl)amino]quinazolin-7-yl}oxy)propyl](ethyl)amino}ethyl dihydrogen phosphate, or a pharmaceutically acceptable salt thereof.

In one embodiment the Aurora kinase inhibitor is a maleate co crystal of AZD1152. The maleate co-crystal of AZD1152 may be synthesised according to the processes described in WO2007/132227.

In one embodiment the Aurora kinase inhibitor is a maleate co crystal of 2-{[3-({4-[(5-{2-[(3-fluorophenyl)amino]-2-oxoethyl}-1H-pyrazol-3-yl)amino]quinazolin-7-yl}oxy)propyl](ethyl)amino}ethyl dihydrogen phosphate. The maleate co-crystal of 2-{[3-({4-[(5-{2-[(3-fluorophenyl)amino]-2-oxoethyl}-1H-pyrazol-3-yl)amino]quinazolin-7-yl}oxy)propyl](ethyl)amino}ethyl dihydrogen phosphate may be synthesised according to the processes described in WO2007/132227.

In another embodiment the Aurora kinase inhibitor may inhibit gene expression, for example by interfering with mRNA stability or translation. In one embodiment the Aurora kinase inhibitor is selected from for example siRNA or shRNA.

It may be convenient or desirable to prepare, purify, and/or handle a corresponding salt of the inhibitor, for example, a pharmaceutically-acceptable salt. A suitable pharmaceutically-acceptable salt of a MEK inhibitor or an Aurora kinase inhibitor may be, for example, an acid-addition salt which is sufficiently basic, for example an acid-addition salt with an inorganic or organic acid. Such acid-addition salts include but are not limited to, furmarate, methanesulfonate, hydrochloride, hydrobromide, citrate and maleate salts and salts formed with phosphoric and sulfuric acid. A suitable pharmaceutically-acceptable salt of a MEK inhibitor or an Aurora kinase inhibitor may be, for example, a salt which is sufficiently acidic, for example an alkali or alkaline earth metal salt. Such alkali or alkaline earth metal salts include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

In one embodiment the MEK inhibitor, or a pharmaceutically acceptable salt thereof, may be linked to the Aurora kinase inhibitor, or a pharmaceutically acceptable salt thereof.

The therapeutic combination, combination product or kit of parts of the present invention is expected to produce a synergistic or beneficial effect through the production of an anti-cancer effect in a patient, which is accordingly useful in the treatment of cancer in a patient. A beneficial effect is achieved if the effect is therapeutically superior, as measured by, for example, the extent of the response, the response rate, the time to disease progression or the survival period, to that achievable on dosing one or other of the components of the combination treatment at its conventional dose. The beneficial effect may be synergistic, if the combined effect is therapeutically superior to the sum of the individual effect achievable with a MEK inhibitor or an Aurora kinase inhibitor. Further, a beneficial effect is obtained if an effect is achieved in a group of patients that does not respond (or responds poorly) to an antagonist of the biological activity of a MEK inhibitor or an Aurora kinase inhibitor alone. In addition, the effect is defined as affording a beneficial effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, a beneficial effect is deemed to be achieved if a conventional dose of a MEK inhibitor or an Aurora kinase inhibitor may be reduced without detriment to one or more of the extent of the response, the response rate, the time to disease progression and survival data, in particular without detriment to the duration of the response, but with fewer and/or less troublesome side-effects than those that occur when conventional doses of each component are used.

Anti-cancer effects which are accordingly useful in the treatment of cancer in a patient include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. It is expected that when a therapeutic combination, combination product or kit of parts of the present invention is administered to a patient in need of treatment for cancer, said therapeutic combination, combination product or kit of parts will produce an effect, as measured by, for example, one or more of: the extent of the anti-tumour effect, the response rate, the time to disease progression and the survival rate. Anti-cancer effects include prophylactic treatment as well as treatment of existing disease.

Pharmaceutically acceptable compositions of the invention comprising an inhibitor with a pharmaceutically acceptable adjuvant, diluent or carrier, may be in a form suitable for oral use (for example as tablets, capsules, aqueous or oily suspensions, emulsions or dispersible powders or granules), for topical use (for example as creams, ointments, gels, or aqueous or oily solutions or suspensions; for example for use within a transdermal patch), for parenteral administration (for example as a sterile aqueous or oily solution or suspension for intravenous, subcutaneous, intramuscular or intravascular dosing) or as a suppository for rectal dosing. In other embodiments the compositions may be delivered endoscopically, intratracheally, intralesionally, percutaneously, intravenously, subcutaneously, intraperitoneally or intratumourally. In general the compositions described herein may be prepared in a conventional manner using conventional excipients or carriers that are well known in the art.

The pharmaceutically acceptable compositions of the invention may be obtained by conventional procedures using conventional pharmaceutically acceptable adjuvants, diluents or carriers that are well known in the art.

Suitable pharmaceutically-acceptable diluents or carriers for a tablet formulation include, for example, inert diluents such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Pharmaceutically acceptable compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin or olive oil.

The dosage of the MEK inhibitor and/or the Aurora kinase inhibitor for a given patient will be determined by the attending physician, taking into consideration various factors known to modify the action of drugs including severity and type of disease, body weight, sex, diet, time and route of administration, other medications and other relevant clinical factors. Therapeutically effective dosages may be determined by either in vitro or in vivo methods.

Sequential administration of the MEK inhibitor and the Aurora kinase inhibitor may be advantageous in enabling both inhibitors to be administered at the dose intended for single use, in contrast to simultaneous administration wherein the dosage of either or both inhibitors would possibly have to be reduced.

The therapeutically effective amount of a MEK inhibitor or an Aurora kinase inhibitor, as described herein, to be used will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage of a MEK inhibitor might range from about 1 mg to up to 250 mg or more, depending on the factors mentioned above. A typical daily dosage of AZD1152, or a pharmaceutically acceptable salt thereof might range from about 1 mg up to 450 mg or more, and will depend upon the schedule of administration and other factors mentioned above. Typically, the clinician will administer the therapeutic combination, combination product or kit of parts until a dosage is reached that achieves the desired effect. Where separate pharmaceutically acceptable compositions are administered, the sequence in which the MEK inhibitor, or pharmaceutically acceptable salt thereof, and the Aurora kinase inhibitor, or pharmaceutically acceptable salt thereof, may be administered (i.e. whether and at what point sequential administration takes place) may be determined by the physician or skilled person.

The dosages and schedules described herein may be varied according to the particular disease state and the overall condition of the patient. For example, it may be necessary or desirable to reduce the above-mentioned doses of the components of the combination treatment in order to reduce toxicity. Dosages and schedules may also vary if, in addition to a therapeutic combination, combination product or kit of parts treatment of the present invention, one or more additional chemotherapeutic agents are used. Scheduling can be determined by the practitioner who is treating any particular patient using his professional skill and knowledge.

The therapeutic combination, combination product or kit of parts of the present invention is expected to be particularly useful for the treatment patients with cancers, including, but not limited to, non-solid tumours such as leukaemia, for example acute myeloid leukaemia, multiple myeloma, haematologic malignancies or lymphoma, and also solid tumours and their metastases such as melanoma, non-small cell lung cancer, glioma, hepatocellular (liver) carcinoma, glioblastoma, carcinoma of the thyroid, cholangiocarcinoma, bile duct, bone, gastric, brain/CNS, head and neck, hepatic, stomach, prostate, breast, renal, testicular, ovarian, cervix, skin, cervical, lung, muscle, neuronal, oesophageal, bladder, lung, uterine, vulval, endometrial, kidney, colon, colorectal, pancreatic, pleural/peritoneal membranes, salivary gland, epidermoid tumours and haematological malignancies.

The therapeutic combination, combination product, or kit of parts as hereinbefore described is expected to be especially useful for the treatment patients with lung cancer, melanoma, colorectal cancer, breast cancer, ovarian cancer, thyroid cancer, pancreatic cancer, prostate cancer, liver cancer, and their metastases, and also for the treatment of patients with leukaemia, such as acute myeloid leukaemia, or multiple myeloma. The therapeutic combination, combination product or kit of parts of the present invention is also expected to be particularly useful for the treatment of patients with a tumour which is associated with the Ras-Raf-MEK-ERK pathway or which is dependent alone, or in part, on the biological activity of the Ras-Raf-MEK-ERK pathway.

The therapeutic combination, combination product or kit of parts of the present invention is also expected to be particularly useful for the treatment of patients with a tumour which is associated with MEK or which is dependent alone, or in part, on the biological activity of MEK.

The therapeutic combination, combination product or kit of parts of the present invention is also expected to be particularly useful for the treatment of patients with a tumour which is associated with Aurora kinase or which is dependent alone, or in part, on the biological activity of Aurora kinase.

The therapeutic combination, combination product or kit of parts of the present invention may be used as a sole therapy or may involve surgery or radiotherapy or an additional chemotherapeutic agent or a therapeutic antibody in addition.

Such chemotherapeutic agents may include one or more of the following categories of anti tumor agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and bosutinib (SKI-606), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Where the administration is sequential or separate, the delay in administering the additional chemotherapeutic agent or therapeutic antibody should not be such as to lose the beneficial effect of the combination.

The following terms, unless otherwise indicated, shall be understood to have the following meanings:

An inhibitor may be a polypeptide, nucleic acid, carbohydrate, lipid, small molecular weight compound, an oligonucleotide, an oligopeptide, siRNA, antisense, a recombinant protein, an antibody, a peptibody, or conjugates or fusion proteins thereof. For a review of siRNA see Milhavet O, Gary D S, Mattson M P. (Pharmacol Rev. 2003 December; 55(4):629-48. For a review of antisense see Opalinska J B, Gewirtz A M. Sci STKE. 2003 Oct. 28; 2003 (206): pe47.

A small molecular weight compound refers to a compound with a molecular weight of less than 2000 Daltons, 1000 Daltons, 700 Daltons or 500 Daltons.

A patient is any warm-blooded animal, such as a human.

The term treatment includes therapeutic and/or prophylactic treatment.

An aurora kinase inhibitor possesses aurora kinase inhibitory activity and in particular Aurora A and/or Aurora B kinase inhibitory activity.

The MEK inhibitor AZD6244 can be prepared according to the process described in International Patent Publication Number WO2003/077914, in particular according to the process described in Example 10. The AZD6244 hydrogen sulphate salt can be prepared according to the process described in International Patent Publication Number WO2007/076245.

The MEK inhibitor 4-(4-Bromo-2-fluorophenylamino)-N-(2-hydroxyethoxy)-1,5-dimethyl-6-oxo-1,6-dihydropyridazine-3-carboxamide can be prepared according to the following method

Step A: Preparation of diethyl 2-(2-methylhydrazono)malonate: To a solution of diethyl ketomalonate (95 g, 546 mmol) in EtOH (600 mL) (2 L 3-neck flask equipped with thermocouple, ° C. (internal temperature, heated by a heating mantle) and stirred for 6 hours. The reaction mixture was cooled to room temperature and stirred overnight. The reaction mixture was concentrated under reduced pressure to give the crude material along with solid precipitates that was purified by a silica gel plug (3:2 hexanes:EtOAc) to afford 81 g (74%) of the desired product. N₂ line, condenser and mechanical stirrer) was added MeNHNH₂ (32 mL, 600 mmol) in one portion at room temperature. The reaction mixture was warmed to 60

Step B: Preparation of diethyl 2-(2-methyl-2-propionylhydrazono)malonate: To a solution of 2-(2-methylhydrazono)malonate (100 g, 494 mmol) in THF (1 L) at 0° C. was added LiHMDS (643 mL, 643 mmol) by an addition funnel over 45 minutes. The reaction mixture was stirred for 45 minutes at 0° C. Propionyl chloride (51.6 mL, 593 mmol) was added in one portion). The resulting mixture was warmed to room temperature and stirred for 20 hours. The reaction mixture was quenched with saturated aqueous NH₄Cl (85 mL) and water (85 mL). The reaction mixture was concentrated under reduced pressure and additional water (300 mL) was added. The resulting mixture was extracted with EtOAc (3×250 mL). The combined organic layers were washed with saturated aqueous NaHCO₃ (2×250 mL) followed by brine (250 mL), dried over MgSO₄, filtered, and concentrated under reduced pressure to give 112 g (88%) of the crude product that was used directly in the next step without further purification.

Step C: Preparation of 4-hydroxy-1,5-dimethyl-6-oxo-1,6-dihydropyridazine-3-carboxylic acid: To a solution of LiHMDS (331 mL, 331 mmol, 1 M solution in THF) in THF (430 mL) at −78° C. was added a solution of 2-(2-methyl-2-propionylhydrazono)malonate (21.40 g, 82.86 mmol) in THF (10 mL). The resulting mixture was slowly warmed to −40° C. over 1 hour and stirred for 1.5 hours at −40° C. To the reaction mixture was added water (500 mL) at −40° C. The reaction mixture was warmed to room temperature and stirred for 3 hours. The reaction mixture was concentrated under reduced pressure, quenched with 6 N aqueous HCl at 0° C., and acidified to pH 1 to 2. The resulting mixture was stirred for 16 hours at room temperature. The precipitates were filtered off and triturated with CH₂Cl₂ to afford 7.21 g (47%) of the desired product. The filtrate was extracted with EtOAc (3×). The combined organic layers were washed with water, dried over MgSO₄, filtered, and concentrated under reduced pressure to give the crude material that was triturated with CH₂Cl₂ to afford additional 3.56 g (23%) of the desired product. The aqueous layer was extracted again with EtOAc (3×). The combined organic layers were washed with water, dried over MgSO₄, filtered, and concentrated under reduced pressure to give the crude material that was triturated with CH₂Cl₂ to afford additional 1.32 g (9%) of the desired product. A total of 12.09 g (79%) of the desired product was obtained.

Step D: Preparation of 4-chloro-1,5-dimethyl-6-oxo-1,6-dihydropyridazine-3-carboxylic acid: A mixture of 4-hydroxy-1,5-dimethyl-6-oxo-1,6-dihydropyridazine-3-carboxylic acid (35.4 g, 192 mmol), catalytic amount of DMF (3 drop), and POCl₃ (178 mL, 1.92 mol) was heated for 2 days at 90° C., and then the POCl₃ was removed under reduced pressure. The crude material was quenched with ice, and the reaction mixture was stirred for 2 hours at room temperature. The precipitates formed out of the solution was filtered off and washed with ether. The precipitates collected were triturated with ether to afford 11.7 g (30%) of the desired product. The filtrate was extracted with EtOAc (2×). The combined organic layers were dried over MgSO₄, filtered, and concentrated under reduced pressure to give the crude product that was triturated with ether and dried under reduced pressure to afford additional 9.56 g (24%) of the desired product. A total of 21.29 g (55%) of the desired product was obtained.

Step E: Preparation of 4-(4-bromo-2-fluorophenylamino)-1,5-dimethyl-6-oxo-1,6-dihydropyridazine-3-carboxylic acid: To a solution of 4-bromo-2-fluoroaniline (22.6 g, 116 mmol) in THF (165 mL) at −78° C. was slowly added a solution of LiHMDS (174 mL, 174 mmol, 1 M solution in THF). The resulting mixture was stirred for 1 hour at −78° C. To this mixture was added 4-chloro-1,5-dimethyl-6-oxo-1,6-dihydropyridazine-3-carboxylic acid (11.0 g, 54.4 mmol) as a solid at −78° C. The reaction mixture was slowly warmed to room temperature and stirred for 21 hour. The reaction was quenched and acidified with 10% aqueous HCl (250 mL) at 0° C. To this mixture was added water (100 mL), EtOAc (350 mL), and brine (50 mL). The reaction mixture was warmed to room temperature and stirred for 30 minutes. The organic layer was separated and the acidic aqueous layer was extracted with EtOAc (2×300 mL). The combined organic layers were dried over MgSO₄, filtered, and concentrated under reduced pressure to give the crude material that was triturated with ether (5×), filtered, washed with ether, and dried under reduced pressure to afford 14.51 g (75%) of the desired product.

Step F: Preparation of 4-(4-bromo-2-fluorophenylamino)-1,5-dimethyl-6-oxo-N-(2-(vinyloxy)ethoxy)-1,6-dihydropyridazine-3-carboxamide: To a suspension of 4-(4-bromo-2-fluorophenylamino)-1,5-dimethyl-6-oxo-1,6-dihydropyridazine-3-carboxylic acid (14.51 g, 40.74 mmol) and HOBt (11.01 g, 81.48 mmol) in DMF (165 mL) was added EDCI (15.62 g, 81.48 mmol) at room temperature. The resulting mixture was stirred for 1.5 hours. O-(2-(Vinyloxy)ethyl)hydroxylamine (8.36 mL, 81.48 mmol) and TEA (11.36 mL, 81.48 mmol) was added to the activated ester at room temperature. After stirring for 1.5 hours, the reaction mixture was diluted with EtOAc and washed with saturated aqueous NH₄Cl, brine, saturated aqueous NaHCO₃ (2×), and brine. The organic layer was separated, dried over MgSO₄, filtered, and concentrated under reduced pressure to give the crude product that was used directly without further purification.

Step G: Preparation of 4-(4-bromo-2-fluorophenylamino)-N-(2-hydroxyethoxy)-1,5-dimethyl-6-oxo-1,6-dihydropyridazine-3-carboxamide: A mixture of 4-(4-bromo-2-fluorophenylamino)-1,5-dimethyl-6-oxo-N-(2-(vinyloxy)ethoxy)-1,6-dihydropyridazine-3-carboxamide (17.98 g, 40.75 mmol) and 6 N aqueous HCl (13.58 mL, 81.50 mmol) in EtOH/THF (50 mL/50 mL) was stirred for 3 hours at room temperature. The reaction mixture was concentrated under reduced pressure and diluted with water (50 mL). The resulting mixture was extracted with EtOAc (2×). The combined organic layers were dried over MgSO₄, filtered, and concentrated under reduced pressure to give the crude material that was purified by silica gel flash column chromatography (100% CH₂Cl₂ to 2.5% MeOH in CH₂Cl₂) to afford 9.41 g (56% for two steps) of the desired product. MS APCI (−) m/z 413, 415 (M−1, Br pattern) detected; ¹H NMR (400 MHz, CD₃OD) δ 7.38 (dd, 1H), 7.27 (d, 1H), 6.79 (t, 1H), 3.99 (t, 2H), 3.80 (s, 3H), 3.74 (t, 2H), 1.77 (s, 3H).

The MEK inhibitor 2-(2-fluoro-4-iodophenylamino)-N-(2-hydroxyethoxy)-1,5-dimethyl-6-oxo-1,6-dihydropyridine-3-carboxamide can be prepared according to the following method

Step A. Preparation of 2-chloro-6-oxo-1,6-dihydro-pyridine-3-carboxylic acid: 2-Chloro-6-oxo-1,6-dihydro-pyridine-3-carboxylic acid was prepared from dichloronicotinic acid (3.00 g, 15.6 mmol, Aldrich) according to the procedure described in U.S. Pat. No. 3,682,932 to yield 1.31 g (48%) of the desired product.

Step B. Preparation of 2-chloro-1-methyl-6-oxo-1,6-dihydro-pyridine-3-carboxylic acid methyl ester: To a solution of 2-chloro-6-oxo-1,6-dihydro-pyridine-3-carboxylic acid (0.644 g, 3.71 mmol) in DMF (20 mL) was added lithium hydride (95%, 0.078 g, 9.28 mmol) and the reaction mixture was stirred for 40 minutes under N₂. Methyl iodide (0.508 mL, 1.16 g, 8.16 mmol) was then added and the reaction mixture was stirred for an additional 45 minutes. The reaction mixture was quenched with 2 M HCl until the pH was 6-7. The reaction mixture was diluted with EtOAc and saturated NaCl and the layers separated. The aqueous layer was back extracted with EtOAc (1×). The combined organic layers were dried (Na₂SO₄) and concentrated under reduced pressure to yield a crude yellow solid. HPLC analysis showed two products in a 4:1 ratio that were separated by flash column chromatography (methylene chloride/EtOAc, 15:1 to 10:1) to give 0.466 g (62%) pure desired product as a white crystalline solid.

Step C. Preparation of methyl 5-bromo-2-chloro-1-methyl-6-oxo-1,6-dihydropyridine-3-carboxylate: To a solution of methyl 2-chloro-1-methyl-6-oxo-1,6-dihydropyridine-3-carboxylate (0.100 g, 0.496 mmol) in DMF (5 mL) was added N-bromosuccinimide (0.177 g, 0.992 mmol) and the reaction mixture was stirred for 4 hours at room temperature under N₂. The reaction mixture was quenched with saturated sodium bisulfite and then diluted with EtOAc and H₂O and the layers separated. The aqueous layer was back extracted with EtOAc (2×). The combined organic layers were dried (Na₂SO₄) and concentrated under reduced pressure to yield a yellow solid in quantitative yield.

Step D. Preparation of methyl 2-chloro-1,5-dimethyl-6-oxo-1,6-dihydropyridine-3-carboxylate: To a suspension of methyl 5-bromo-2-chloro-1-methyl-6-oxo-1,6-dihydropyridine-3-carboxylate (0.400 g, 1.43 mmol) and 1,1′-bis(diphenylphosphino)ferrocenedichloropalladium(II) (0.0587 g, 0.0713 mmol) in dioxane (8 mL) at 0° C. under N₂ was added dimethylzinc (0.713 mL, 1.43 mmol, 2 M solution in toluene). The reaction mixture was immediately heated to 100° C. for 30 minutes. The reaction mixture was cooled to 0° C. and quenched with MeOH (0.800 mL). The reaction mixture was diluted with EtOAc and washed with 1 M HCl. The aqueous layer was back extracted with EtOAc (1×). The combined organic layers were washed with saturated NaCl, dried (Na₂SO₄) and concentrated under reduced pressure to a dark yellow gum. Purification by flash column chromatography (methylene chloride/EtOAc, 15:1) gave 0.164 g (53%) pure desired product as a yellow crystalline solid.

Step E: Preparation of methyl-(2-fluoro-4-iodophenylamino)-1,5-dimethyl-6-oxo-1,6-dihydropyridine-3-carboxylate: To a solution of 2-fluoro-4-iodobenzenamine (0.058 g, 0.31 mmol) in THF (2 mL) at −78° C. under N₂ was added lithium bis(trimethylsilyl)amide (0.56 mL, 0.56 mmol, 1 M solution in hexanes) dropwise. The reaction mixture was stirred for one hour at −78° C. Methyl 2-chloro-1,5-dimethyl-6-oxo-1,6-dihydropyridine-3-carboxylate (0.060 g, 0.28 mmol) was then added dropwise as a solution in THF (1 mL) and the reaction mixture was stirred for 25 minutes at −78° C. The reaction mixture was quenched by the addition of H₂O and the pH was adjusted with 0.1M HCl and then diluted with EtOAc and saturated NaCl and the layers separated. The aqueous layer was back extracted with EtOAc (1×). The combined EtOAc layers were dried (Na₂SO₄) and concentrated under reduced pressure. Purification by flash column chromatography (methylene chloride/EtOAc, 20:1) gave 0.086 g (84%) pure desired product as a white crystalline solid. MS ESI (+) m/z 417 (M+1) detected; ¹H NMR (400 MHz, CDCl₃) δ 9.56 (s, 1H), 7.79 (s, 1H), 7.49 (d, 1H), 7.36 (d, 1H), 6.43 (t, 1H), 3.85 (s, 3H), 3.30 (s, 3H), 2.15 (s, 3H).

Step F: Preparation of 2-(2-fluoro-4-iodophenylamino)-1,5-dimethyl-6-oxo-N-(2-(vinyloxy)ethoxy)-1,6-dihydropyridine-3-carboxamide: To a solution of methyl 2-(2-fluoro-4-iodophenylamino)-1,5-dimethyl-6-oxo-1,6-dihydropyridine-3-carboxylate (0.500 g, 1.20 mmol) in THF (60 mL) was added O-(2-vinyloxy-ethyl)-hydroxylamine (0.149 g, 1.44 mmol). The solution was cooled to 0° C. and lithium bis(trimethylsilyl)amide (4.81 ml, 4.81 mmol) (1 M solution in hexanes) was added dropwise. The reaction mixture was warmed to room temperature. After stirring for 10 minutes the reaction mixture was quenched by the addition of 1 M HCl and partitioned between EtOAc and saturated NaCl. The layers were separated and the organic layer was dried (Na₂SO₄) and concentrated under reduced pressure to yield a crude yellow solid that was used without purification in the next step.

Step G: Preparation of 2-(2-fluoro-4-iodophenylamino)-N-(2-hydroxyethoxy)-1,5-dimethyl-6-oxo-1,6-dihydropyridine-3-carboxamide: To a solution of crude 2-(2-fluoro-4-iodophenylamino)-1,5-dimethyl-6-oxo-N-(2-(vinyloxy)ethoxy)-1,6-dihydropyridine-3-carboxamide (0.585 g, 1.20 mmol) in ethanol (10 mL) was added aqueous 2 M HCl (3 mL). The reaction mixture was stirred for 45 minutes at room temperature. The pH of the reaction mixture was adjusted to pH 7 with 1 M NaOH. The reaction mixture was diluted with EtOAc and H₂O. The organic layer was separated and washed with saturated NaCl. The combined aqueous layers were back extracted with EtOAc (1×). The combined organic layers were dried (Na₂SO₄) and concentrated under reduced pressure. Purification by silica gel flash column chromatography (methylene chloride/MeOH, 15:1) gave 2-(2-fluoro-4-iodophenylamino)-N-(2-hydroxyethoxy)-1,5-dimethyl-6-oxo-1,6-dihydropyridine-3-carboxamide (0.421 g; 76% over two steps) as a pale yellow solid. MS ESI (+) m/z 462 (M+1) pattern detected; ¹H NMR (400 MHz, CDCl₃) δ 9.77 (s, 1H), 8.50 (s, 1H), 7.47 (d, 1H), 7.36 (d, 1H), 6.43 (t, 1H), 4.04 (br s, 2H), 3.85 (br s, 1H), 3.74 (br s, 2H), 3.29 (s, 3H), 2.14 (s, 3H). MS ESI (+) m/z 462 (M+1) pattern detected.

The invention will now be illustrated by the following non-limiting examples, which are provided for illustrative purposes only and are not to be construed as limiting upon the teachings herein, in which:

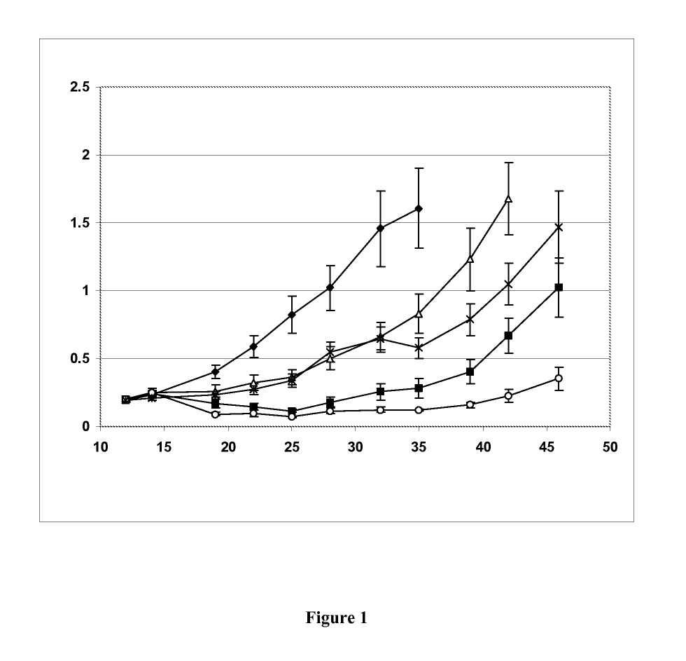

FIG. 1. Shows combination sequence effects of AZD1152 and AZD6244 on the growth of Calu-6 tumours in female nude mice; mean tumour volume (cm³) measured at days post tumour innoculation in mice. Closed diamonds represent the control; closed squares represent AZD1152 alone; open triangles represent AZD6244 alone; open circles represent administration of AZD1152 followed by AZD6244; crosses represent administration of AZD6244 followed by AZD1152.

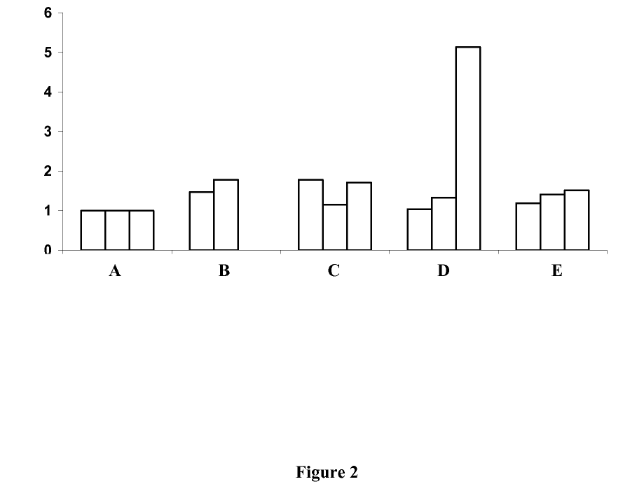

FIG. 2. Flow cytometry analysis of tumours to assess effects on proportion of cells in G₂-M phase (G2M), Polyploidy (PP) and Cell Death (Sub G1) following dosing of AZD1152 followed by AZD6244 dose sequence. The data bars represent fold increase in the parameters compared to control groups. In each 3 bar group the left bar represents G2M, the middle bar represents PP and the right bar represents Sub G1. A=control; B=AZD1152; C=AZD1152 followed by 24 hr recovery; D=AZD1152 followed by 24 hr recovery followed by AZD6244; E=AZD6244.

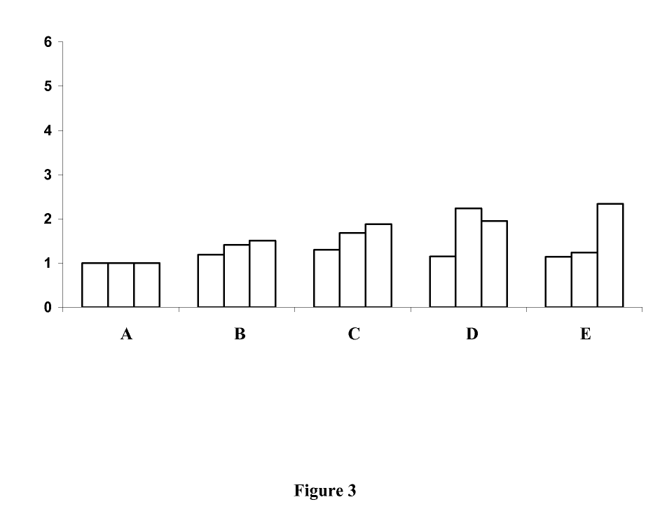

FIG. 3. Flow cytometry analysis of tumours to assess effects on proportion of cells in G₂-M phase (G2M), Polyploidy (PP) and Cell Death (Sub G1) following dosing of AZD6244 followed by AZD1152 dose sequence. The data bars represent fold increase in the parameters compared to control groups. In each 3 bar group the left bar represents G2M, the middle bar represents PP and the right bar represents Sub G1. A=control; B=AZD6244; C=AZD6244 followed by 24 hr recovery; D=AZD6244 followed by 24 hr recovery followed by AZD1152; E=AZD1152.

### EXAMPLE 1

### In Vivo Combination Study of AZD1152 and AZD6244 in the Calu-6 Human Lung Cancer Xenograft Model

To establish the in vivo efficacy of combination therapy of AZD1152 and AZD6244 in Calu-6 human lung xenograft model, experiments were conducted in female athymic mice (Swiss nu/nu genotype, ≧6 weeks of age). Calu-6 human lung tumour xenografts were established in mice by injecting 1×10⁶ cells (100 μl volume containing 50% Matrigel®) subcutaneously in the dorsal flank. Tumour volumes were assessed using bilateral Vernier calliper measurement at least twice weekly and calculated using the formula (length×width)×√(length×width)×(π/6), where length was taken to be the longest diameter across the tumour and width the corresponding perpendicular. Mice were randomised into 4 treatment groups (10 mice/group) when the mean tumour volume reached approximately 0.2 cm³. Following randomisation, mice were treated with drug vehicles for AZD1152 or AZD6244, AZD1152, AZD6244 or a combination of AZD1152 plus AZD6244 according to treatment schedules described below:

### Schedule A:

Animals were dosed for 2 days with either drug vehicle for AZD1152 (0.3M Tris Buffer at pH 9) or AZD1152 (150 mg/kg/day) administered as a continuous subcutaneous infusion. A 24 h period was allowed to lapse after the end of AZD1152 dosing, after which either drug vehicle for AZD6244 (H.P.M.C. (0.5% w/v Methocel/0.1% w/v Tween 80)) or AZD6244 (25 mg/kg administered twice daily) was dosed orally for 14 days.

### Schedule B:

Animals were dosed for 14 days with either drug vehicle for AZD6244 (H.P.M.C. (0.5% w/v Methocel/0.1% w/v Tween 80)) or AZD6244 (25 mg/kg administered orally twice daily). A 24 h period was allowed to lapse after the end of AZD6244 dosing, after which drug vehicle for AZD1152 (0.3M Tris Buffer at pH 9) or AZD1152 (150 mg/kg/day) was dosed as a continuous subcutaneous infusion for 2 days.

Tumour growth inhibition was assessed by comparison of the differences in tumour volume between control and treated groups on Day 35 of the study. The effects of combination treatment sequence were assessed by comparing any effect on tumour growth in the group of animals receiving AZD1152 and AZD6244 with tumour growth in the groups where animals received single agent therapy alone. Sub-groups of animals were sacrificed from each treatment group at the end of AZD1152, AZD6244 or combination dosing to assess the pharmacodynamic effects on tumours using Flow Cytometry.

AZD1152 and AZD6244, when dosed as monotherapy, produced a significant growth inhibition of Calu-6 tumours compared to the vehicle-dosed group (94.7%, p<0.0005 & 57.8%, p<0.005 respectively). When dosed in combination, the two therapies produced an inhibition of tumour growth (105.1%; p<0.0005 for AZD1152+AZD6244 sequence & 74.4%; p<0.0005 for AZD6244+AZD1152 sequence) compared to the vehicle-dosed group (FIG. 1). Additionally, the AZD1152+AZD6244 sequence showed increased anti-tumour efficacy compared to AZD1152 monotherapy (195.57%; p<0.005) and the AZD6244+AZD1152 sequence showed increased anti-tumour efficacy compared to AZD6244 monotherapy (39.24%; p<0.05). The combination sequence of AZD1152 first followed by AZD6244 produced a greater inhibition of tumour growth than the AZD6244 first followed by AZD1152 sequence (114.8%, p<0.0005).

In summary, administering AZD1152 before AZD6244 showed greater anti-tumour response compared to administering AZD6244 before AZD1152.

Flow cytometry analysis showed a greater proportion of cells in Sub G1 phase (an indication of levels of apoptosis) in the combination group where AZD1152 was dosed first compared to the combination group where AZD6244 was dosed first (compared to the control groups there was 5.13 and 1.95 fold increase respectively).

1. A therapeutic combination comprising
a MEK inhibitor, or a pharmaceutically acceptable salt thereof, and
an Aurora kinase inhibitor, or a pharmaceutically acceptable salt thereof.

2. A combination product comprising
a MEK inhibitor, or a pharmaceutically acceptable salt thereof, and
an Aurora kinase inhibitor, or a pharmaceutically acceptable salt thereof, in association with a pharmaceutically acceptable adjuvant, diluent or carrier.

3. A kit of parts comprising the following components
a MEK inhibitor, or a pharmaceutically acceptable salt thereof, in association with a pharmaceutically acceptable adjuvant, diluent or carrier; and
an Aurora kinase inhibitor, or a pharmaceutically acceptable salt thereof, in association with a pharmaceutically acceptable adjuvant, diluent or carrier,
wherein the components are provided in a form which is suitable for sequential, separate and/or simultaneous administration.

4. A therapeutic combination according to claim 1, a combination product according to claim 2 or a kit of parts according to claim 3, wherein the MEK inhibitor is 6-(4-Bromo-2-chloro-phenylamino)-7-fluoro-3-methyl-3H-benzoimidazole-5-carboxylic acid (2-hydroxy-ethoxy)-amide, or a pharmaceutically acceptable salt thereof.

5. A therapeutic combination according to claim 1, a combination product according to claim 2 or a kit of parts according to claim 3, wherein the MEK inhibitor is 2-(2-fluoro-4-iodophenylamino)-N-(2-hydroxyethoxy)-1,5-dimethyl-6-oxo-1,6-dihydropyridine-3-carboxamide, or a pharmaceutically acceptable salt thereof

6. A therapeutic combination according to claim 1, a combination product according to claim 2 or a kit of parts according to claim 3, wherein the MEK inhibitor is 4-(4-Bromo-2-fluorophenylamino)-N-(2-hydroxyethoxy)-1,5-dimethyl-6-oxo-1,6-dihydropyridazine-3-carboxamide, or a pharmaceutically acceptable salt thereof.

7. A therapeutic combination according to claim 1, a combination product according to claim 2 or a kit of parts according to claim 3, wherein the Aurora kinase inhibitor is 2-{[3-({4-[(5-{2-[(3-fluorophenyl)amino]-2-oxoethyl}-1H-pyrazol-3-yl)amino]quinazolin-7-yl}oxy)propyl] (ethyl)amino}ethyl dihydrogen phosphate, or a pharmaceutically acceptable salt thereof.

8. A therapeutic combination according to claim 1, a combination product according to claim 2 or a kit of parts according to claim 3, wherein the MEK inhibitor is 6-(4-Bromo-2-chloro-phenylamino)-7-fluoro-3-methyl-3H-benzoimidazole-5-carboxylic acid (2-hydroxy-ethoxy)-amide, or a pharmaceutically acceptable salt thereof and the Aurora kinase inhibitor is 2-{[3-({4-[(5-{2-[(3-fluorophenyl)amino]-2-oxoethyl}-1H-pyrazol-3-yl)amino]quinazolin-7-yl}oxy)propyl](ethyl)amino}ethyl dihydrogen phosphate, or a pharmaceutically acceptable salt thereof.

9. A method of treating cancer, comprising administration of a therapeutically effective amount of a MEK inhibitor, or a pharmaceutically acceptable salt thereof, in combination with an Aurora kinase inhibitor, or a pharmaceutically acceptable salt thereof, to a patient having, or suspected of having, cancer.

10. A method of treating cancer according to claim 9, wherein one or more doses of the Aurora kinase inhibitor, or a pharmaceutically acceptable salt thereof, are administered prior to the administration of one or more doses of the MEK inhibitor, or a pharmaceutically acceptable salt thereof.

11. A method of treating cancer according to claim 10 wherein there is an interval between the administration of one or more doses of the Aurora kinase inhibitor, or a pharmaceutically acceptable salt thereof, and the administration of one or more doses of the MEK inhibitor, or a pharmaceutically acceptable salt thereof.

12. A method of treating cancer according to claim 11 wherein the interval is 24 hours.

13. A method of treating cancer according to any one of claims 9 to 12, wherein the cancer is selected from lung cancer, melanoma, colorectal cancer, breast cancer, ovarian cancer, thyroid cancer, pancreatic cancer, prostate cancer, liver cancer, acute myeloid leukaemia or multiple myeloma.
